(19) United States
(12) Patent Application Publication
(10) Pub. No.: US 2025/0025832 A1
(43) Pub. Date: Jan. 23, 2025

(54) METHOD FOR ADAPTING AN NH3 DOSING AMOUNT AND AN NH3 FILL LEVEL DISTRIBUTION OF AN SCR CATALYST FOR AN SCR EXHAUST GAS AFTERTREATMENT SYSTEM WITH AT LEAST ONE SCR CATALYST

(71) Applicant: Robert Bosch GmbH, Stuttgart (DE)

(72) Inventors: Arman Khosravani, Freiberg Am Neckar (DE); Stefanos Tzivanakis, Waiblingen (DE); Thorsten Meier, Pfinztal-Kleinsteinbach (DE)

(21) Appl. No.: 18/777,506

(22) Filed: Jul. 18, 2024

(30) Foreign Application Priority Data
Jul. 21, 2023 (DE) 10 2023 206 939.3

(51) Int. Cl. B01D 53/94; G01N 33/00

(52) U.S. Cl. CPC B01D 53/9418; B01D 53/9477; G01N 33/0014; G01N 33/0031; G01N 33/0037; B01D 2257/404; B01D 2258/012

(57) ABSTRACT

Method for adapting an NH3 dosing amount and an NH3 fill level distribution of an SCR catalyst (22) for an SCR exhaust gas aftertreatment system (25) having at least one SCR catalyst (22).

### BACKGROUND

DE 10 2019 216 520 A1 relates to a method for adjusting the dosing amount of a reducing agent for an SCR catalyst, comprising: determining (110) an expected temperature profile in at least an axial portion of the catalyst (70) for a predetermined time period (tSim); first simulating (120) the resulting amount of reducing agent behind the at least one portion of the catalyst with a first predetermined amount of reducing agent dosing as a function of the determined expected temperature profile; comparing the first simulated amount of reducing agent to a threshold value; depending on the result of the comparison, selecting a second predetermined dosing amount and second simulating (130, 160) a resulting amount of reducing agent behind the at least a portion of the catalyst (70) with the second dosing amount; comparing the second simulated amount of reducing agent to the threshold value; and adjust (140, 150, 170, 180, 190, 195) the dosing amount for injecting the reducing agent into the catalyst based on the first and/or the second comparison.

DE 10 2016 201 602 A1 relates to a method for determining an ammonia mass flow between two SCR catalysts arranged in succession in an exhaust gas line in an SCR catalyst system, which comprises only one reducing agent dosing unit upstream of the first SCR catalyst, characterized in that the determination is made from the signal of a NOx sensor arranged between the two SCR catalysts and the signal of a NOx sensor arranged downstream of the second SCR catalyst.

DE 10 2021 213 316 A1 relates to a method for estimating an ammonia concentration (xNH3est) downstream of at least one SCR catalyst (22) arranged in an exhaust gas line (11) in an SCR system (20) of an internal combustion engine (10), which comprises at least one reducing agent dosing unit (21) upstream of the first SCR catalyst (22), a first NOx sensor (31) is arranged upstream of the SCR catalyst (22), which determines a first NOx signal (xsens, US), wherein a second NOx sensor (32) is arranged downstream of the SCR catalyst (22), which determines a second NOx signal (xsens,DS) wherein a release for the method is granted, when an operating state for the internal combustion engine (10) is detected, and based on the granted release, a start time (t0) for the estimation of the ammonia concentration (xNH3est) is determined, the estimation of the ammonia concentration (xNH3est) is determined from the first and second NOx signals (xsens, US,xsens, DS) and an efficiency model (ηNOxmdl) and the SCR system (20) is adapted as a function of the estimated ammonia concentration (xNH3est).

### SUMMARY

In a first aspect, the invention relates to a method for adapting an NH3 dosing amount and an NH3 fill level distribution of an SCR catalyst for an SCR exhaust gas aftertreatment system having at least one SCR catalyst, wherein the at least one SCR catalyst is divided into at least two bricks, wherein a first NOx sensor signal is determined by means of a first NOx sensor signal upstream of the SCR catalyst and downstream of the internal combustion engine, wherein a second NOx sensor signal downstream of the SCR catalyst is determined by means of a second NOx sensor, wherein the first and second NOx sensors have a transverse sensitivity to ammonia, wherein a division of the second NOx sensor signal into a modeled NOx mass flow and a modeled NH3 mass flow control unit is determined by means of the model stored on the control unit, wherein an evaluation window is started as a function of a first criterion and ended as a function of a second criterion, wherein a cumulative modeled NH3 mass flow is determined via the evaluation window and a first cumulative NH3 mass flow is determined by means of an SCR model, wherein an overdosing model is determined based on the SCR model, which receives a virtually increased NH3 dosing amount compared to the SCR model, wherein a second cumulative NH3 mass flow is determined by means of the overdosing model, wherein an underdosing model is determined based on the SCR model, which receives a reduced NH3 dosing amount compared to the SCR model, wherein a third cumulative NH3 mass flow rate is determined by means of the underdosing model, wherein an adaptation value between the modeled NH3 mass flow and the first NH3 mass flow is determined by means of a predeterminable control unit, wherein an adaptation of an NH3 dosing amount and/or an adaptation of the fill level distribution of the bricks of the SCR catalyst is performed as a function of the cumulative modeled NH3 mass flows and the cumulative second and third NH3 mass flow.

The method has the particular advantage that in defined operating situations, an evaluation of the SCR state can be performed with the aid of the ammonia amount (NH3 slip) stored from the SCR catalyst. The goal of the method is to correct the dosing amount and/or deviations of the modeled fill level distribution of the SCR catalyst via an adaptation factor. This is to ensure that NOx and NH3 emissions can be met below defined limits.

It is further advantageous that a control to the maximum NOx conversion level is possible by the method. Thus, the SCR catalyst is placed on its NH3 slip side.

The method is therefore suitable for zero NOx emission SCR control. System tolerances due to installed components or aging effects of the components of the SCR system can be compensated for, especially on average. Furthermore, deviations to NH3 models can be checked and adapted.

In a particular embodiment, an exhaust gas mass flow, an exhaust gas pressure, an exhaust gas temperature, an oxygen concentration downstream of the internal combustion engine and upstream of the SCR catalyst, and dosing amount information are determined as input variables for the guide model, the overdosing model, and the underdosing model.

In a further embodiment, if the cumulative modeled NH3 mass flow exceeds the second cumulative NH3 mass flow, an adaptation of the NH3 dosing amount is performed based on the adaptation value, in particular a reduction of the NH3 dosing amount is performed.

Thus, an adjustment of an overdose of ammonia may be counteracted, resulting in reduced NH3 slip from the SCR catalyst.

In an advantageous embodiment, if the cumulative modeled NH3 mass flow falls below the third cumulative NH3 mass flow, an adaptation of the NH3 dosing amount is performed based on the adaptation value, in particular an increase of the NH3 dosing amount is performed.

It is advantageous here that an underdosing of ammonia can thus be counteracted so that an optimal NOx conversion can be performed.

In an alternative embodiment, a first, second and third fill level amount is determined as a function of the SCR model, the overdosing model and the underdosing model for the at least two bricks of the SCR catalyst, wherein an approximation function is determined as a function of the cumulative first, second and third NH3 gas flow and the first, second and third fill level amount for each brick, wherein a target NH3 fill level is determined by means of an approximation function and the cumulative modeled NH3 mass flow for each brick, and an adaptation of the fill level amounts of the at least two bricks of the SCR catalyst is performed as a function of the target NH3 fill level, in particular to adapt the SCR model, the overdosing model and the underdosing model. The adaptation of the SCR model, in particular the guide model, the overdosing model and the underdosing model, has the particular advantage that a correction of the modeled fill level distribution can be performed. This ensures a continuous improvement of the SCR model and emissions can be compensated for even better.

In a further embodiment, if the cumulative modeled NH3 mass flow exceeds the first cumulative NH3 mass flow, an adaptation of the fill level amounts is performed as a function of the first fill level amounts and the second fill level amounts, in particular linearly.

Thus, the NH3 fill level distribution for the SCR catalyst is improved and provides a more precise prediction of the fill level amount.

In a particular embodiment, if the cumulative modeled NH3 mass flow falls below the first cumulative NH3 mass flow, an adaptation is performed as a function of the first fill level amounts and the third fill level amounts, in particular linearly.

Thus, the NH3 fill level distribution for the SCR catalyst is improved and provides a more precise prediction of the fill level amount.

In an alternative embodiment, if the modeled NH3 concentration exceeds a predeterminable first NH3 threshold value, the first criterion is satisfied, and if the modeled NH3 concentration falls below the predeterminable first NH3 threshold value, the second criterion is satisfied.

This is advantageous because ammonia can neither be modeled continuously well enough, nor can it be reliably measured below certain concentration levels.

In a particular embodiment, if the first NH3 mass flow exceeds a predeterminable first NH3 threshold value, the first criterion is satisfied, and if the first NH3 mass flow falls below the predeterminable first NH3 threshold value, the second criterion is satisfied.

This is advantageous because ammonia can neither be modeled continuously well enough, nor can it be reliably measured below certain concentration levels.

In an alternative embodiment, instead of a modeled NH3 mass flow, an NH3 mass flow sensor value of the NH3 sensor is used.

In further aspects, the invention relates to a device, in particular a control device and a computer program configured, in particular programmed, to carry out any one of the methods. In yet another aspect, the invention relates to a machine-readable storage medium on which the computer program is stored.

### BRIEF DESCRIPTION OF THE DRAWINGS

The invention will be explained in more detail in the following with reference to an exemplary embodiment shown in the figures.

Shown are:

FIG. 1 a schematic illustration of an internal combustion engine having an exhaust gas aftertreatment system according to the invention; and

FIG. 2 is an exemplary measurement for the method presented,

FIG. 3 a flow chart for graphically representing the flow of an exemplary embodiment of the method.

### DETAILED DESCRIPTION

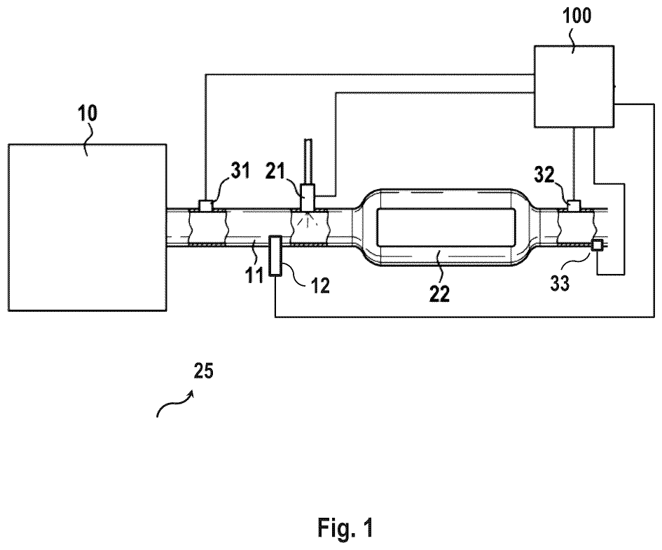

An internal combustion engine 10 has an SCR exhaust aftertreatment system 25 with at least one SCR catalyst 22 in its exhaust gas line 11, which is shown in FIG. 1. This has a reducing agent dosing unit 21 with which a urea solution (AdBlue) can be injected into the exhaust gas line 11. Ammonia is released from this at the high temperatures of the exhaust gas. A first SCR catalyst 22 is arranged downstream of the reducing agent dosing unit 21. Further SCR catalysts may be arranged downstream of the first SCR catalyst 22. A first NOx sensor 31 and a temperature sensor 12 are arranged upstream of the reducing agent dosing unit 21 and downstream of an internal combustion engine 10 in the exhaust gas line 11. The first NOx sensor 31 measures a first NOx concentration sensor value NOx1, preferably as an NOx concentration or as an NOx mass flow. A second NOx sensor 32 is arranged downstream of the first SCR catalyst 22 and thereby measures a second NOx concentration sensor value NOx2, preferably as an NOx concentration or as an NOx mass flow.

This also applies in particular to the NH3 concentration or an NH3 mass flow.

In an optional configuration, an NH3 sensor 33 may further be installed downstream of SCR catalyst 22. The NH3 sensor may thereby determine an NH3 mass flow.

All NOx sensors 31, 32 relay their signals to an electronic control unit 100. As NOx sensors 31, 32 are also cross-sensitive to ammonia in addition to nitrogen oxides, their signals are sum signals of nitrogen oxides and ammonia. However, the first NOx sensor 31 is arranged upstream of the reducing agent dosing unit 21 so that it reliably only measures the nitrogen oxide amount in the exhaust gas. The reducing agent dosing unit 21 also reports the amount of ammonia dosed into exhaust gas line 11 to the control unit 100.

Model N1 is also stored on the control unit 100, which can determine an NOx concentration component and an NH3 concentration component for the sum signal of the second NOx sensor 32 as a function of the second NOx concentration sensor value NOx2.

However, the described model N1 does not provide continuous NOx and NH3 concentration components and is based on the second NOx concentration sensor value NOx2 downstream of the SCR catalyst 22. Optionally, the exhaust gas temperature of temperature sensor 12 and/or the first NOx concentration sensor value NOx1 may be used for the determination.

The model N1 can also be used to determine an NOx concentration component and an NH3 concentration component for the sum signal of the first NOx sensor 31 as a function of the first NOx concentration sensor value NOx1.

Furthermore, an SCR model M1, in particular NH3 guide model, is stored on the control unit 100 for the SCR catalyst 22, which divides the SCR catalyst 22 into n virtual bricks, with n=2 . . . i, iε [symbol] . In so doing, SCR catalyst 22 is modeled using a multi-slice model, wherein the current fill level amount F_{M1i} is determined for each virtual brick n. Preferably, the fill level amounts correspond to NH3 fill level amounts.

Furthermore, an exhaust gas mass flow \dot{m}ₑₓₕ, an exhaust gas pressure pₑₓₕ, an exhaust gas temperature Tₑₓₕ, an oxygen concentration downstream of the internal combustion engine 10 and upstream of the SCR catalyst 22, and dosing amount information are determined.

In particular, depending on the determined exhaust gas temperature Tₑₓₕ, a brick temperature T_{exh,n} is determined for each brick n of the SCR catalyst 22 by means of a temperature model stored on the control unit 100 and used as an input variable for the SCR model M1.

Furthermore, an overdosing model M2 is determined based on the SCR model M1, which is assigned a virtually increased NH3 dosing amount as the input variable compared to the SCR model M1.

In an advantageous configuration, the overdosing model M2 is assigned dosing amount information that is, for example, 10% higher than the SCR model M1 as an input variable. The overdosing model M2 is modeled using a multi-slice model, wherein the current fill level amount F_{M2i} is determined for each virtual brick n.

Furthermore, an underdosing model M3 is determined based on the SCR model M1, which is assigned a virtually decreased NH3 dosing amount as the input variable compared to the SCR model M1.

In an advantageous configuration, the underdosing model M3 is assigned dosing amount information that is 10% lower than the SCR model M1 as the input variable. The underdosing model M3 is modeled using a multi-slice model, wherein the current fill level amount F_{M3i} is determined for each virtual brick.

Furthermore, a dosing strategy is stored on the control unit 100, which determines a dosing amount as a function of, for example, an exhaust gas mass flow \dot{m}ₑₓₕ, a temperature of an SCR catalyst 22, the NOx concentrations upstream and downstream of the SCR catalyst 22, and an ammonia level for the SCR catalyst 22.

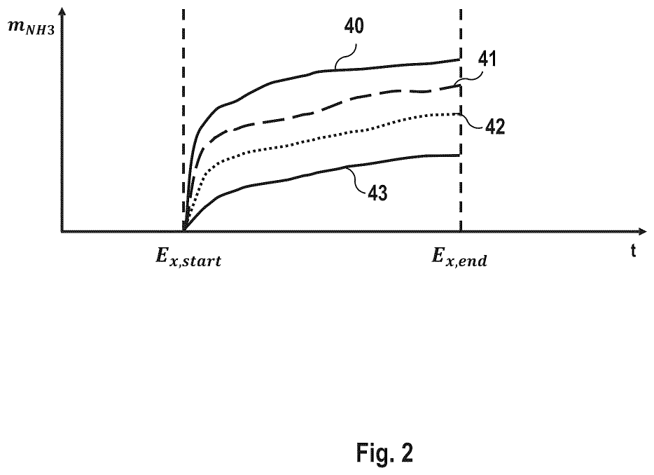

FIG. 2 shows an exemplary measurement for the method presented.

The NH3 mass m_{NH3}, preferably in grams, is depicted on the ordinates and the time t on the abscissa. The start of an evaluation window Eₓ is shown at a first time E_{x,start} and an end of the evaluation window Eₓ is shown at a second time E_{x,end}.

A total of four NH3 mass flows 40;41;42;43 are shown.

The first NH3 mass 40 corresponds to the second NH3 mass flow NH3_{M2} integrated via the evaluation window Eₓ, which is determined by means of the overdosing model M2.

The second NH3 mass 41 corresponds to the modeled NH3 mass flow NH3/1 integrated via the evaluation window Eₓ, which is determined by means of the model N1 from the second NOx concentration sensor value NOx2.

The third NH3 mass 42 corresponds to the first NH3 mass flow NH3_{M1} integrated via the evaluation window Eₓ, which is determined by means of the SCR model M1.

The fourth NH3 mass 43 corresponds to the third NH3 mass flow NH3_{M3} integrated via the evaluation window Eₓ, which is determined by means of the underdosing model M3.

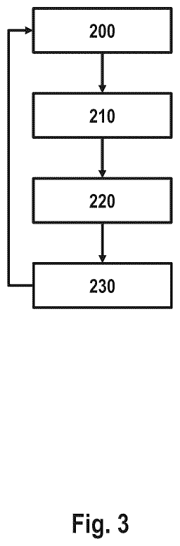

FIG. 3 shows the exemplary method sequence for adapting an NH3 dosing amount and an NH3 fill level distribution of an SCR catalyst 22 for an SCR exhaust gas aftertreatment system having at least one SCR catalyst 22 using a flow diagram.

In a first step 200, a first and second NOx sensor signal NOx1; NOx2 is continuously received and stored by the control unit 100.

By means of a model N₁ stored on the control unit 100, a division into a modeled NH3 mass flow NH3_{M1} and a NOx mass flow NOx_{N1} is determined as a function of the second NOx sensor signal NOx2. The modeled NH3 and modeled NOx concentration component corresponds to an NH3 and NOx concentration signal downstream of SCR catalyst 22, in particular at the location of second NOx sensor 32.

Furthermore, an SCR model M1 for the SCR catalyst 22 is stored on the control unit 100, which divides the SCR catalyst 22 into n virtual bricks, with n=2 . . . i, iε [symbol] . The SCR catalyst 22 is modeled using a multi-slice model,
wherein the current fill level amount Fhd M1i is determined for each virtual brick n. Preferably, the fill level amounts correspond to NH3 fill level amounts.

Furthermore, an exhaust gas mass flow \dot{m}ₑₓₕ, an exhaust gas pressure pₑₓₕ, an exhaust gas temperature Tₑₓₕ, an oxygen concentration downstream of the internal combustion engine 10 and upstream of the SCR catalyst 22, and dosing amount information are determined.

In particular, depending on the determined exhaust gas temperature Tₑₓₕ, a brick temperature T_{exh,n} is determined for each brick n of the SCR catalyst 22 by means of a temperature model stored on the control unit 100 and used as an input variable for the SCR model M1.

Furthermore, an overdosing model M2 is determined based on the SCR model M1, which is assigned a virtually increased NH3 dosing amount as the input variable compared to the SCR model M1.

In an advantageous configuration, the overdosing model M2 is assigned dosing amount information that is, for example, 10% higher than the SCR model M1 as an input variable. The overdosing model M2 is modeled using a multi-slice model, wherein the current fill level amount F_{M2i} is determined for each virtual brick n.

Furthermore, an underdosing model M3 is determined based on the SCR model M1, which is assigned a virtually decreased NH3 dosing amount as the input variable compared to the SCR model M1.

In an advantageous configuration, the underdosing model M3 is assigned dosing amount information that is 10% lower than the SCR model M1 as the input variable. The underdosing model M3 is modeled using a multi-slice model, wherein the current fill level amount F_{M3i} is determined for each virtual brick. The method then continues in a step 210.

In a step 210, an evaluation window Eₓ is started as a function of a first criterion and ended as a function of a second criterion.

In an advantageous embodiment, the NH3 concentration NH3_{M1} is monitored by the control unit 100.

If the modeled NH3 concentration NH3_{M1} exceeds a predeterminable first threshold value S_{NH3}, a start of the evaluation window Eₓ is detected and if the modeled NH3 concentration NH3_{M1} falls below the predeterminable first threshold S_{NH3}, the evaluation window Eₓ is ended again.

In an alternative embodiment, a first NH3 mass flow NH3_{M1} is monitored by the control unit 100.

If the first NH3 mass flow NH3_{M1} exceeds a predeterminable first NH3 threshold value S_{NH3}, the start of the evaluation window Eₓ is detected and if the first NH3 mass flow NH3_{M1} falls below the predeterminable first NH3 threshold value S_{NH3}, an end of the evaluation window is detected.

The method may proceed directly to detecting the start of the evaluation window Eₓ in step 220, or alternatively to detecting the end of the evaluation window Eₓ in step 220.

In a step 220, a cumulative modeled NH3 mass flow NH3_{M1}, Eₓ, a first cumulative NH3 mass flow NH3_{M1}, Eₓ, a second cumulative NH3 mass flow NH3_{M2}, Eₓ and a third cumulative NH3 mass flow NH3_{M3}, Eₓ are determined by the control unit 100 in the evaluation window Eₓ.

In so doing, the first cumulative NH3 mass flow NH3_{M1}, Eₓ is determined by means of the SCR model M1 stored on the control unit 100 as a function of an exhaust gas mass flow \dot{m}ₑₓₕ, an exhaust gas pressure pₑₓₕ, an exhaust gas temperature _{Texh}, or the brick temperatures T_{exh,n}, an oxygen concentration downstream of the internal combustion engine 10 and upstream of the SCR catalyst 22 and dosing amount information.

The SCR model M1 is also referred to as an NH3 guide model M1.

Furthermore, an adaptation value NH3_{Adaption} between the modeled NH3 mass flow NH3_{M1} and the first NH3 mass flow NH3_{M1} is determined by means of a predeterminable controller, in particular an integral controller or a virtual controller.

The method may then continue in a step 230.

In a step 230, it is checked whether the determined adaptation value NH3_{Adaption} IS above the second cumulative NH3 mass flow NH3_{M2}, Eₓ, below the third cumulative NH3 mass flow NH3_{M3}, Eₓ or between the second and third cumulative NH3 mass flows NH3_{M2},Eₓ,NH3_{M3},Eₓ.

If the cumulative modeled NH3 mass flow NH3_{M1}, Eₓ exceeds the second cumulative NH3 mass flow NH3_{M2},Eₓ, an adaptation of the NH3 dosing amount is performed based on the adaptation value NH3_{Adaption}.

In this case, in particular, an overdosing of ammonia for the SCR system is detected and the NH3 dosing amount for the dosing strategy is reduced accordingly.

If the cumulative modeled NH3 mass flow NH3_{M1}, Eₓ falls below the third cumulative NH3 mass flow NH3_{M3}, Eₓ, an adaptation of the NH3 dosing amount is performed based on the adaptation value NH3_{Adaption}.

In this case, in particular, an underdosing of ammonia for the SCR system is detected and the NH3 dosing amount for the dosing strategy is increased accordingly.

If the cumulative modeled NH3 mass flow NH3_{M1}, Eₓ the second cumulative NH3 mass flow NH3_{M2}, Eₓ is between the second cumulative NH3 mass flow NH3_{M2}, Eₓ and the third cumulative NH3 mass flow NH3_{M3},Eₓ, the first, second and third level amounts F_{M1i}, F_{M2i} and F_{M3i} for the at least two bricks of the SCR catalytic converter 22 are determined using the SCR model M1, the overdosing model M2 and the underdosing model M3.

An approximation function A; is then determined as a function of the cumulative first, second and third NH3 mass flows NH3_{M1}, Eₓ; NH3_{M2}, Eₓ; NH3_{M3}, Eₓ and the first second and third fill level amounts F_{M1i}, F_{M2i}, F_{M3i} for the at least two bricks. In particular, linear interpolation is used for this purpose.

In the event that the cumulative modeled NH3 mass flow NH3_{M1}, Eₓ exceeds the first cumulative NH3 mass flow NH3_{M1}, Eₓ, an adaptation of the fill level amounts F_{M1i}, F_{M2i}, F_{M3i} is performed as a function of the first fill level amounts F_{M1i} and the second fill level amounts F_{M2i}, in particular linearly.

For this purpose, the cumulative modeled NH3 mass flow NH3_{M1}, Eₓ is used in the approximation functions Aᵢ and a target NH3 fill level Fᵢ, NH3_{N1} for the at least two bricks of the SCR catalyst 22 is obtained.

The adaptation of the fill level amounts F_{M1i}, F_{M2i}, F_{M1i} of the at least two bricks of the SCR catalyst 22 takes place as a function of the target NH3-fill level Fᵢ, NH3_{N1}, in particular for the adaptation of the SCR model M1, the overdosing model M2 and the underdosing model M3.

The method can then be started from the beginning in step 200 and terminated.

In the event that the cumulative modeled NH3 mass flow NH3_{M1}, Eₓ falls below the first cumulative NH3 mass flow NH3_{M1}, Eₓ, an adaptation of the fill level amounts F_{M1i}, F_{M2i}, F_{M1i} is performed as a function of the first fill level amounts Fhd M1i and the third fill level amounts F_{M3i}, in particular linearly.

For this purpose, the cumulative modeled NH3 mass flow NH3_{M1}, Eₓ is used in the approximation functions A; and a target NH3 fill level Fᵢ, NH3_{N1} for the at least two bricks of the SCR catalyst 22 is obtained.

The adaptation of the fill level amounts F_{M1i}, F_{M2i}, F_{M3i} of the at least two bricks of the SCR catalyst 22 takes place as a function of the target NH3 fill level Fᵢ, NH3_{N1}, in particular for the adaptation of the SCR model M1, the overdosing model M2 and the underdosing model M3.

The method can then be started from the beginning in step 200 and terminated.

1. A method for adapting an NH3 dosing amount and an NH3 fill level distribution of an SCR catalyst (22) for an SCR exhaust gas aftertreatment system (25) having at least one SCR catalyst (22), wherein the at least one SCR catalyst (22) is divided into at least two bricks,
wherein a first NOx sensor signal (NOx1) upstream of the SCR catalyst (22) and downstream of the internal combustion engine (10) is determined by means of a first NOx sensor (31),
wherein a second NOx sensor (32) is used to determine a second NOx sensor signal (NOx2) downstream of the SCR catalyst (22),
wherein the first and second NOx sensors (31, 32) have a transverse sensitivity to ammonia (NH3),
wherein a division of the second NOx sensor signal (NOx2) into a modeled NOx mass flow (NOx_{N1}) and a modeled NH3 mass flow (NH3_{M1}) is determined by means of a model (N1) stored on the control unit (100),
wherein an evaluation window (E_{X}) is started as a function of a first criterion and ended as a function of a second criterion,
wherein a cumulative modeled NH3 mass flow (NH3_{M1}, E_{X}) is determined via the evaluation window (E_{X}) and a first cumulative NH3 mass flow (NH3_{M1},E_{X}) is determined by means of an SCR model (M1),
wherein an overdosing model (M2) is determined based on the SCR model (M1) which receives an increased NH3 dosing amount compared to the SCR model (M1),
wherein a second cumulative NH3 mass flow (NH3_{M2}, E_{X}) is determined by means of the overdosing model (M2),
wherein an overdosing model (M3) is determined based on the SCR model (M1) which receives a reduced NH3 dosing amount compared to the SCR model (M1),
wherein a third cumulative NH3 mass flow (NH3_{M3}, E_{X}) is determined by means of the underdosing model (M3),
wherein an adaptation value (NH3_{Adaption}) between the modeled NH3 mass flow (NH3_{N1}) and the first NH3 mass flow (NH3_{M1}, E_{X}) is determined by means of a predeterminable controller, and
wherein, an adaptation of an NH3 dosing amount and/or an adaptation of the fill level distribution of the bricks of the SCR catalyst (22) is performed as a function of the cumulative modeled NH3 mass flows (NH3_{M1},E_{X}) and the cumulative second and third NH3 mass flow (NH3_{M2}, E_{X}, NH3_{M3}, E_{X}).

2. The method according to claim 1, wherein an exhaust gas mass flow (\dot{m}ₑₓₕ), an exhaust gas pressure (pₑₓₕ), an exhaust gas temperature (Tₑₓₕ), an oxygen concentration downstream of the internal combustion engine (10) and upstream of the SCR catalyst (22) and dosing amount information are determined as input variables for the guide model (M1), the overdosing model (M2) and the underdosing model (M3).

3. The method according to claim 1, wherein when the cumulative modeled NH3 mass flow (NH3_{N1},E_{X}) exceeds the second cumulative NH3 mass flow (NH3_{M2}, E_{X}), an adaptation of the NH3 dosing amount is performed based on the adaptation value (NH3_{Adaption}).

4. The method according to claim 1, wherein if the cumulative modeled NH3 mass flow (NH3_{N1},E_{X}) falls below the third cumulative NH3 mass flow (NH3_{M3}, E_{X}), an adaptation of the NH3 dosing amount is performed based on the adaptation value (NH3_{Adaption}).

5. The method according to claim 1, wherein a first, second and third fill level amount (F_{M1i}, F_{M2i}, F_{M3i}) is determined as a function of the SCR model (M1), the overdosing model (M2) and the underdosing model (M3) for the at least two bricks of the SCR catalyst (22), wherein an approximation function (Ai) is determined as a function of the cumulative first, second and third NH3 gas flow (NH3_{M1},Eₓ;NH3_{M2},Eₓ;NH3_{M3},Eₓ) and the first, second and third fill level amount (F_{M1i}, F_{M2i}, F_{M3i}) for each brick, wherein a target NH3 fill level (Fᵢ, NH3_{M1}) is determined by means of an approximation function (Aᵢ) and the cumulative modeled NH3 mass flow (NH3_{N1}, E_{X}) for each brick, and an adaptation of the fill level amounts (F_{M1i}, F_{M2i}, F_{M3i}) of the at least two bricks of the SCR catalyst (22) is performed as a function of the target NH3 fill level (Fᵢ, NH3_{N1}).

6. The method according to claim 5, wherein if the cumulative modeled NH3 mass flow (NH3_{N1},E_{X}) exceeds the first cumulative NH3 mass flow (NH3_{M1}, E_{X}), an adaptation of the fill level amounts (F_{M1i}, F_{M2i}, F_{M3i}) is performed in each case as a function of the first fill level amounts (F_{M1i}) and the second fill level amounts (F_{M2i}), linearly.

7. The method according to claim 5, wherein if the cumulative modeled NH3 mass flow (NH3_{N1},E_{X}) falls below the first cumulative NH3 mass flow (NH3_{M1},E_{X}), an adaptation of the fill level amounts (F_{M1i}, F_{M2i}, F_{M3i}) is performed in each case as a function of the first fill level amounts (F_{M1i}) and the third fill level amounts (F_{M3i}), linearly.

8. The method according to claim 1, wherein if the modeled NH3 concentration (NH3_{N1}) exceeds a predeterminable first NH3 threshold value (S_{NH3}), the first criterion is satisfied, and if the modeled NH3 concentration (NH3/1) falls below the predeterminable first NH3 threshold value (S_{NH3}), the second criterion is satisfied.

9. The method according to claim 1, wherein if the first NH3 mass flow (NH3_{M1}) exceeds a predeterminable first NH3 threshold value (S_{NH3}), the first criterion is satisfied, and if the first NH3 mass flow (NH3_{M1}) falls below the predeterminable first NH3 threshold value (S_{NH3}), the second criterion is satisfied.

10. The method according to claim 1, wherein instead of a modeled NH3 mass flow (NH3_{N1}), an NH3 mass flow sensor value of the NH3 sensor (33) is used.

11. A non-transitory, computer-readable medium containing instructions that when executed by a computer cause the computer to adapt an NH3 dosing amount and an NH3 fill level distribution of an SCR catalyst (22) for an SCR exhaust gas aftertreatment system (25) having at least one SCR catalyst (22), wherein the at least one SCR catalyst (22) is divided into at least two bricks,
by determining a first NOx sensor signal (NOx1) upstream of the SCR catalyst (22) and downstream of the internal combustion engine (10) via a first NOx sensor (31),
by determining a second NOx sensor signal (NOx2) downstream of the SCR catalyst (22) via a second NOx sensor (32),
wherein the first and second NOx sensors (31, 32) have a transverse sensitivity to ammonia (NH3),
by providing a division of the second NOx sensor signal (NOx2) into a modeled NOx mass flow (NOx_{N1}) and determining a modeled NH3 mass flow (NH3_{N1}) via a model (N1) stored on the control unit (100),
by starting an evaluation window (E_{X}) is started as a function of a first criterion and ended as a function of a second criterion,
by determining a cumulative modeled NH3 mass flow (NH3_{M1}, E_{X}) via the evaluation window (E_{X}) and a first cumulative NH3 mass flow (NH3_{M1},E_{X}) is determined via an SCR model (M1),
by determining an overdosing model (M2) based on the SCR model (M1) which receives an increased NH3 dosing amount compared to the SCR model (M1),
by determining a second cumulative NH3 mass flow (NH3_{M2}, E_{X}) via of the overdosing model (M2),
by determining an overdosing model (M3) based on the SCR model (M1) which receives a reduced NH3 dosing amount compared to the SCR model (M1),
by determining a third cumulative NH3 mass flow (NH3_{M3}, E_{X}) by means of the underdosing model (M3),
by determining an adaptation value (NH3_{Adaption}) between the modeled NH3 mass flow (NH3_{N1}) and the first NH3 mass flow (NH3_{M1}, E_{X}) via a predeterminable controller, and
by adapting an NH3 dosing amount and/or adapting the fill level distribution of the bricks of the SCR catalyst (22) as a function of the cumulative modeled NH3 mass flows (NH3_{N1}, E_{X}) and the cumulative second and third NH3 mass flow (NH3_{M2}, Eₓ,NH3_{M3}, E_{X}).

12. A control unit (100), which is configured to perform a method according to claim 1.
